(19) United States
(12) Patent Application Publication
(10) Pub. No.: US 2014/0249340 A1
(43) Pub. Date: Sep. 4, 2014

(54) PROCESS FOR THE PRODUCTION OF MIDDLE DISTILLATES FROM A FEEDSTOCK COMPRISING BUTANOL AND PENTANOL

(71) Applicant: IFP ENERGIES NOUVELLES, Rueil-Malmaison Cedex (FR)

(72) Inventors: Vivien Tom, Lyon (FR); Vincent Coupard, Villeurbanne (FR); Francois Hugues, Vernaison (FR); Raphael Huyghe, Saint Andeol Le Chateau (FR)

(73) Assignee: IFP ENERGIES NOUVELLES, Rueil-Malmaison Cedex (FR)

(21) Appl. No.: 14/195,882

(22) Filed: Mar. 4, 2014

(30) Foreign Application Priority Data
Mar. 4, 2013 (FR) 13/51886

(51) Int. Cl. C07C 2/08; C07C 5/05

(52) U.S. Cl. CPC C07C 2/08; C07C 5/05
USPC 585/255

(57) ABSTRACT

The invention relates to a process for the production of middle-distillate hydrocarbon-containing bases from a feedstock comprising butanol and pentanol, with said process comprising at least:
a) Isomerizing dehydration of said feedstock;
b) Separation of the water that is present in said olefinic effluent;
c) Purification of the organic liquid effluent coming from b);
d) Selective oligomerization of a feedstock that comprises at least a portion of the purified organic effluent coming from c), to produce a first oligomerization effluent;
e) Oligomerization of said first oligomerization effluent in such a way as to produce a second oligomerization effluent;
f) Fractionation of said second oligomerization effluent into at least three products, a light product, an intermediate product and a distillate product;
g) Oligomerization of at least a portion of said intermediate product;
h) Hydrogenation of at least a portion of said distillate product.

### TECHNICAL FIELD OF THE INVENTION

This invention relates to the transformation of alcohols comprising 4 to 5 carbon atoms, and more particularly bioalcohols comprising 4 to 5 carbon atoms, into a fuel base. It relates more particularly to a flexible catalytic process for transformation of butanol and pentanol into middle distillates, also called kerosene.

### PRIOR ART

The demand for a use of renewable resources as a partial replacement for petroleum resources for the synthesis of fuels continues to grow. Thus, the use of bioalcohols in the synthesis of bases for fuels is gaining more and more active interest.

Bioalcohols are defined as alcohols that are produced from renewable resources coming from biomass, such as, for example, the lignocellulosic raw materials.

The latter are cellulosic materials, i.e., consisting of more than 90% by weight of cellulose, and/or lignocellulosic materials, i.e., consisting of cellulose, hemicelluloses, which are polysaccharides essentially consisting of pentoses and hexoses, as well as lignin, which is a macromolecule of complex structure and high molecular weight, consisting of aromatic alcohols connected by ether bonds.

The emergence of the projects for production of bioalcohols is broadly supported by the public and industrial push to develop second-generation biofuels, over time upgrading a vast array of lignocellulosic raw materials.

By comparison with ethanol, the alcohols comprising 4 to 5 carbon atoms have several advantages: higher energy density; less hydrophilic nature; better compatibility, both with the storage/transport infrastructures of fuels and with existing automobile engines; lower vapor pressures; less corrosive. Isobutanol is particularly compatible with diesel fuel whereas n-butanol is miscible with gasoline. Likewise, n-pentanol can be supplemented with diesel fuel, and the isopentanols are miscible in gasoline, diesel fuel, or kerosene.

N-butanol is one of the products of fermentation called ABE (acetone-butanol-ethanol) of Clostridium acetobutylicum. The recent projects use selected strains, mutant or OGM, making possible a specific production of n-butanol, one of the limiting factors being its toxicity for the microorganisms in question. More tolerant strains and continuous or semi-continuous extraction processes are therefore necessary. The bacteria that are used in these processes are always of the genus Clostridium.

Isobutanol, or 2-methylpropan-1-ol, is a branched isomer of n-butanol. It is also a product of the fermentation of carbohydrates. Its paths of biosynthesis are, however, different from those of n-butanol. The processes for the production of isobutanol are being developed by players such as Gevo or Butamax from bacterial sources that are different from those used for the production of n-butanol (E. coli).

The n-pentanol or 1-pentanol can be produced from acidic fermentation of lignocellulose that produces valeric acid, and then by hydrogenation.

The iso-pentanols are obtained essentially from fusel oils that are a co-product of alcoholic fermentation.

The patent application WO2009/079213 describes the production of bases for fuels from biomass comprising stages of fermentation into alcohol, dehydration of a portion of said alcohols into olefins, oligomerization of a portion of said olefins, and optional hydrogenation of the product of the oligomerization. By contrast, the patent does not propose a means of monitoring the temperature rise in the reactor due to the exothermicity of the oligomerization reaction. The oligomerization is carried out in a single stage.

The patent application WO2011/140560 describes the production of a kerosene base from lignocellulosic raw materials by taking an isobutanol path. Said raw materials are fermented under specific conditions suitable for the production of isobutanol. The latter is next dehydrated and then oligomerized for producing a fraction that is close to kerosene. This application addresses neither the problem of monitoring the temperature rise in the reactor due to the exothermicity of the oligomerization nor the problem of monitoring the composition of the products that are obtained.

The patent FR2975103A1 describes the production of a kerosene or diesel fraction from an olefinic fraction that for the most part contains 4 to 6 carbon atoms. The process that is described can contain a single total oligomerization stage of the feedstock or, in a second version, two distinct oligomerization stages, with the first making essentially the iso-olefins react and the second making the normal olefins react, with the iso-olefins and the olefins produced by the reactions taking place in the first section. This application does not address the problem of monitoring the temperature rise in the oligomerization reactors due to the exothermicity of the reactions.

### OBJECT AND ADVANTAGE OF THE INVENTION

This invention relates to a process for the production of middle-distillate hydrocarbon-containing bases from a feedstock that comprises butanol and pentanol, with said process comprising at least:
a) A stage for isomerizing dehydration of said feedstock in the presence of an amorphous or zeolitic acid catalyst in at least one reactor, operating at an absolute pressure at the reactor inlet of between 0.5 and 2 MPa and at a temperature at the reactor inlet of between 350 and 450° C. in such a way as to produce an effluent that is for the most part olefinic,
b) A stage for separation of the water that is present in said effluent that is for the most part olefinic operating at a pressure of between 0.5 and 1.2 MPa and at a temperature of between 35 and 60° C. in such a way as to obtain at least one organic liquid effluent,
c) A stage for purification of the organic liquid effluent coming from stage b) in such a way as to produce a purified organic effluent,
d) A first stage for selective oligomerization of isobutenes and isopentenes of a feedstock that comprises at least a portion of the purified organic effluent coming from stage c), in the presence of an amorphous catalyst in at least one reactor that operates at an absolute pressure of between 0.5 and 10 MPa, at a temperature at the reactor inlet of between 40 and 95° C., and at an hourly volumetric flow rate of between 0.1 and 10 h⁻¹, in such a way as to produce a first oligomerization effluent, comprising at least 20% by weight of olefins having a number of carbon atoms that is greater than or equal to 6, with the percentage by weight being expressed relative to the total mass of olefins contained in said effluent,
e) A second stage for oligomerization of said first oligomerization effluent, of the entire effluent coming from the oligomerization stage g), and at least a portion of the light product coming from the fractionation stage f), in the presence of an amorphous catalyst in at least one reactor operating at an absolute pressure of between 2 and 15 MPa, at a temperature at the reactor inlet of between 100 and 200° C., and at an hourly volumetric flow rate of between 0.1 and 10 h⁻¹, in such a way as to produce a second oligomerization effluent,
f) A stage for fractionation of said second oligomerization effluent into at least three products that correspond respectively to a light product that for the most part comprises the C₂ to C₅ compounds, an intermediate product that for the most part comprises the C₆ to C₉ compounds, and a middle distillate product that for the most part comprises the compounds that have at least 10 carbon atoms,
g) A third stage for oligomerization of at least a portion of said intermediate product in the presence of an amorphous catalyst in at least one reactor that operates at an absolute pressure of between 2 and 15 MPa, at a temperature of between 100 and 200° C., and at an hourly volumetric flow rate of between 0.1 and 5 h⁻¹,
h) A stage for hydrogenation of at least a portion of said middle distillate product in the presence of a catalyst that comprises at least one metal of group VIII in at least one reactor that operates at an absolute pressure of between 2 and 4 MPa, at a temperature of between 100 and 350° C., and at an hourly volumetric flow rate of between 1 and 5 h⁻¹ with a hydrogen to hydrocarbon H₂/HC molar ratio of between 10 and 450.

One advantage of the invention is a better monitoring of the products that are obtained, by the separation of the two oligomerization stages, with said separation also making it possible to limit the increase in temperature in the reactor due to the exothermicity of the reactions in each of said stages.

Another advantage of the invention is that it is possible to add aromatic compounds to the fuel base that is produced in accordance with the invention up to the limits provided by the standard ASTM D7566-11a while taking into account the density specification of said standard.

### DETAILED DESCRIPTION OF THE INVENTION

### Feedstock

The feedstock that is treated in the process according to the invention is a feedstock that comprises butanol and pentanol, with the term butanol characterizing all of the alcohols comprising 4 carbon atoms and the term pentanol characterizing all of the alcohols comprising 5 carbon atoms. Said feedstock is advantageously a feedstock that comprises biobutanol and biopentanol, i.e., a feedstock that is produced from renewable resources coming from the biomass. Said feedstock for the most part comprises butanol, at a level of more than 25% by weight, and preferably more than 33% by weight; pentanol, with a content of between 25% and 32%; and it also comprises water, at a content of between 0 and 50% by weight, preferably between 0% and 30% by weight, and in a preferred manner between 15% and 25% by weight; a content of cationic impurities such as, for example, the ions Na⁺, Ca²⁺, K⁺, Mn²⁺, Fe²⁺, Cu²⁺, Zn²⁺, advantageously less than 0.1% by weight; a content of anionic impurities such as, for example, the ions of chloride, sulfate, nitrite, nitrate, phosphate, advantageously less than 0.1% by weight; a content of metals such as nickel, chromium, and potassium, advantageously less than 0.1% by weight; a content of other alcohols such as, for example, methanol and ethanol, advantageously less than 10% by weight, and preferably less than 5% by weight; a content of oxidized compounds other than the alcohols, such as, for example, ethers, acids, ketones, aldehydes, acetals and esters, advantageously less than 1% by weight; and a content of nitrogen-containing compounds and sulfur-containing compounds, such as, for example, amines, acetonitriles, nitric sulfates, and carbon sulfide, advantageously less than 0.5% by weight, with the percentages by weight being expressed relative to the total mass of said feedstock.

The process according to the invention advantageously comprises a purification stage that is carried out prior to the dehydration stage a) in such a way as to eliminate the cationic and anionic impurities as well as at least a portion of the oxidized compounds for limiting the deactivation of the dehydration catalyst placed downstream.

Said purification stage is advantageously carried out by means that are known to one skilled in the art, such as, for example, the use of at least one resin, the adsorption of impurities, and oxidized compounds on solids that are selected from among molecular sieves, activated carbon, alumina, and zeolites, and the distillation for producing a purified feedstock comprising butanol and pentanol that responds to levels of impurities that are compatible with the dehydration catalyst and a product that comprises the organic impurities.

Said levels of impurities that are compatible with the dehydration catalyst are a content of ionic impurities of less than 100 ppm, a metal content of less than 50 ppm, a content of oxidized impurities of less than 150 ppm, and a content of nitrogen-containing and sulfur-containing compounds of less than 30 ppm.

A pretreatment stage can also advantageously be carried out by hydrogenation of the oxidized unsaturated compounds in the presence of a nickel-based catalyst, with said pretreatment stage being carried out before or after the purification stage and preferably after.

### Dehydration Stage a)

In accordance with the invention, the feedstock that comprises butanol and pentanol, optionally purified, undergoes an isomerizing dehydration stage a) in such a way as to produce an effluent that is for the most part olefinic, with said stage operating in the presence of a dehydration catalyst that is known to one skilled in the art, in particular an amorphous acid catalyst or a zeolitic acid catalyst in at least one reactor.

An effluent that is for the most part olefinic is defined as an effluent that comprises water and at least 92%, preferably at least 97%, and in a preferred manner at least 98%, by weight of C₄ and C₅ olefins, including between 9% and 90% by mass of C₄ olefins relative to the total mass of the carbon-containing compounds that are present in said effluent that is produced by said stage a). In addition to the majority presence of butenes and pentenes, said carbon-containing effluent can also comprise other compounds that contain hydrocarbons, hydroxycarbons or oxycarbons in a very minor proportion. In particular, said carbon-containing effluent advantageously comprises less than 5%, preferably less than 3%, by weight of compounds having a number of carbon atoms that is greater than or equal to 6, and oxidized compounds, such as, for example, CO₂, CO, diethyl ether and acetaldehyde, with the percentages being expressed in terms of percentages by weight relative to the total mass of the carbon-containing compounds that are present in said effluent that is produced in said stage a).

Said effluent preferably contains, in % by mass relative to the overall product:

Between 1% and 22%, more preferably between 5 and 10%, isobutene,

Between 3% and 55%, more preferably between 10 and 20%, n-butene,

Between 1% and 20%, more preferably between 5 and 8%, isopentene,

Between 3% and 57%, more preferably between 10 and 20%, n-pentene.

Said isomerizing dehydration stage a) makes it possible to convert isobutanol and the branched isomers of pentanol into a mixture of butenes and pentenes.

In the case where the catalyst that is used in the dehydration stage a) is a zeolitic catalyst, said catalyst comprises at least one zeolite that is selected from among the zeolites that have at least pore openings that contain 10 or 12 oxygen atoms (10 MR or 12 MR). Actually, it is known to define the size of the pores of the zeolites by the number of oxygen atoms forming the annular section of the channels of zeolites, called “member ring” or MR in English. In a preferred manner, said zeolitic catalyst comprises at least one zeolite that has a structural type that is selected from among the following structural types: MFI, FAU, MOR, FER, SAPO, TON, CHA, EUO, MEL, MTT and BEA. The zeolites that are described in Zhang et al., Applied Catalysis A: General 403 (2011) 1-11 are also usable.

The zeolite that is used in the catalyst used in stage a) of the process according to the invention can advantageously be modified by dealuminification or desilication according to any dealuminification or desilication method that is known to one skilled in the art.

In the case where the catalyst that is used in the dehydration stage a) is an amorphous acid catalyst, said catalyst comprises at least one porous refractory oxide that is selected from among alumina, alumina activated by a deposit of mineral acid, and silica-alumina.

Said amorphous or zeolitic dehydration catalyst used in stage a) of the process according to the invention can also advantageously comprise at least one oxide-type matrix that is also called a binder. Matrix according to the invention is defined as an amorphous or poorly crystallized matrix.

Said matrix is advantageously selected from among the elements of the group that is formed by clays (such as, for example, among the natural clays such as kaolin and bentonite), magnesia, aluminas, silicas, silica-aluminas, aluminates, titanium oxide, boron oxide, zirconia, aluminum phosphates, titanium phosphates, zirconium phosphates, and carbon. Preferably, said matrix is selected from among the elements of the group that is formed by aluminas, silicas, and clays.

In a preferred mode, the binder has a macroporous texture as described in the patent U.S. Pat. No. 7,880,048.

The dehydration catalyst used in stage a) of the process according to the invention is advantageously shaped in the form of grains of different shapes and sizes. It is advantageously used in the form of extrudates that are cylindrical or multilobed, such as bilobed, trilobed, or polylobed of straight or twisted shape, but it can optionally be manufactured and employed in the form of crushed powder, tablets, rings, balls, wheels, or spheres. Preferably, said catalyst is in the form of extrudates or balls.

The isomerizing dehydration stage a) of the process according to the invention is performed at an absolute pressure at the reactor inlet of between 0.5 and 2 MPa, preferably between 0.6 and 1.1 MPa, and at a temperature of between 350 and 450° C., preferably between 350 and 400° C., more preferably between 350 and 375° C. The hourly volumetric flow rate is between 2 and 7 h⁻¹.

Hourly volumetric flow rate is defined as the volumetric flow rate of the feedstock at the reactor inlet of m³/h at 15° C., 1 atm divided by the volume of catalyst in m³ contained in the reactor.

The absolute pressure at the reactor inlet is selected in such a way that said feedstock of said stage a) is in the gaseous phase at the reactor inlet. The high operating pressure advantageously makes it possible to separate the products from the water by liquid/liquid decanting.

Said stage a) is very endothermic. It is therefore advantageously performed in at least two separate reactors, with the effluent from one reactor being heated before being sent as a feedstock for the next reactor.

The conversion of the feedstock comprising butanol and pentanol in stage a) is advantageously greater than 95%, preferably 99%, and in a preferred manner greater than 99.7%. Conversion of the feedstock comprising butanol and pentanol is defined as the ratio of the difference between the mass flow rate of said feedstock entering stage a) and the mass flow rate of said feedstock leaving stage a) to the mass flow rate of said feedstock entering stage a).

### Stage b) for Separation of Water

In accordance with the invention, the effluent that is for the most part olefinic coming from stage a) undergoes at least one stage b) for separation of the water that is present in said effluent. The water has a negative effect on the catalysts of the subsequent stages of the process according to the invention. The effluents from stage b) are an aqueous liquid effluent, an organic liquid effluent, and optionally a gaseous effluent that comprises carbon monoxide, carbon dioxide, hydrogen, methane, and acetone, taken by itself or in a mixture. Said stage b) is preferably a decanting stage in which an aqueous phase is separated from an organic phase.

Said stage b) is advantageously performed at a pressure of between 0.5 and 1.2 MPa, preferably between 0.6 and 1.1 MPa, and at a temperature of between 35 and 60° C.

The high operating pressure of the dehydration stage a) according to the invention makes it possible to condense the water in stage b) at a high temperature. When the dehydration stage is carried out at a lower pressure, it is either necessary to use a cold cycle to condense the water or necessary to compress the effluent at the outlet of the dehydration stage.

### Purification Stage c)

In accordance with the invention, the organic liquid effluent coming from stage b) undergoes a purification stage c) in such a way as to produce a purified organic effluent.

Said stage c) can advantageously be carried out by any method that is known to one skilled in the art, for example by a treatment in an absorption column with MDEA (methyldiethylamine) or another amine followed by a treatment on a molecular sieve, said sieves advantageously being of the type 13X, 3A, 4A and 5A, taken by itself or in a mixture. Said stage c) can also advantageously be carried out by treatment in a washing column with soda. Said stage c) can also advantageously be carried out by treatment of said organic effluent on a molecular sieve, said sieves advantageously being of the type 13X, 3A, 4A and 5A, taken by itself or in a mixture. Said stage c) can also advantageously comprise a treatment on a base resin in such a way as to remove the alcohols that are present in said organic liquid effluent. Driers can advantageously be used in such a way as to attain a water content that is compatible with the oligomerization catalysts that are used downstream in the oligomerization stages d) and e).

The water content of said purified organic effluent is advantageously between 0 and 1,000 ppm, preferably between 0 and 500 ppm, and in a preferred manner between 0 and 200 ppm. The content of sulfur-containing components, for example H₂S or COS, of said purified organic effluent is advantageously less than 100 ppm, preferably less than 50 ppm. The content of nitrogen-containing components, for example ammonia, of said purified organic effluent is less than 1 ppm. The content of inorganic components of said purified organic effluent is less than 1 ppm, preferably below the detection limit.

### Stage d) for Selective Oligomerization of Isobutenes and Isopentenes Called “Selectopol”

In accordance with the invention, at least a portion—and preferably all—of the purified organic effluent coming from the purification stage c) constitutes the first oligomerization feedstock, with said feedstock undergoing a first stage d) for selective oligomerization of isobutenes and isopentenes in at least one reactor in the presence of an amorphous catalyst in such a way as to produce a first oligomerization effluent.

At least one portion of the purified organic effluent coming from stage c) is defined as at least 50% by weight, preferably at least 90% by weight, and in a preferred manner the entire effluent coming from said stage c), with the percentages by weight being expressed relative to the total mass of said effluent.

The conversion of the isobutene in stage d) is advantageously greater than 80%, preferably greater than 85%, and in a preferred manner greater than 95%. Conversion of the isobutene is defined as the ratio of the difference between the mass flow rate of isobutene entering stage d) and the mass flow rate of isobutene leaving stage d) to the mass flow rate of isobutene entering stage d).

The conversion of n-butenes in stage d) is advantageously less than 15%, preferably less than 10%, and in a preferred manner less than 7%. The conversion of n-butenes is defined in a manner that is analogous to the conversion of isobutene.

The conversion of isopentenes in stage d) is advantageously greater than 60%, preferably greater than 75%, and in a preferred manner greater than 80%. The conversion of isobutenes is defined in a manner that is analogous to the conversion of isobutene.

The conversion of n-pentenes in stage d) is advantageously less than 15%, preferably less than 10%, and in a preferred manner less than 5%. The conversion of the n-pentenes is defined in a manner that is analogous to the conversion of isobutene.

Said first oligomerization effluent comprises at least 20% by weight of olefins having a number of carbon atoms that is greater than or equal to 6, preferably at least 25% by weight, and in a preferred manner at least 28% by weight, with the percentage by weight being expressed relative to the total mass of the olefins contained in said effluent. Among the olefins that have a number of carbon atoms of greater than or equal to 6, the C₈-C₁₆ olefins are in the majority relative to the olefins that have at least 16 carbon atoms, i.e., the mass ratio of the C₈-C₁₆ olefins to the olefins that have at least 16 carbon atoms is greater than 1.

The n-olefins with 4 and 5 carbon atoms do not react much in stage d). Their high content entering stage d) makes it possible to minimize the rise in temperature in the reaction section from stage d) and, by the better monitoring of the exothermicity thus obtained, to improve the conversion yield of olefins having less than 9 carbon atoms.

The catalyst used in the first oligomerization stage d) comprises at least one element of group VIII, preferably selected from among nickel, cobalt, iron, platinum, and palladium, and in a preferred manner said element is nickel, and at least one porous oxide refractory substrate that is preferably selected from among alumina, silica, silica-aluminas, siliceous aluminas, zirconias, titanium oxide, magnesia, the clays taken by themselves or in a mixture, and in a preferred manner said substrate is an alumina or a silica-alumina, preferably a silica-alumina. In a preferred arrangement, the catalyst that is used in the first oligomerization stage d) is a silica-alumina-based catalyst as described in the patent U.S. Pat. No. 7,572,946.

Said catalyst that is used in said stage d) of the process according to the invention also advantageously comprises at least one oxide-type matrix that is also called a binder. According to the invention, matrix is defined as an amorphous or poorly-crystallized matrix.

Said matrix is advantageously selected from among the elements of the group that is formed by clays (such as, for example, the natural clays such as kaolin or bentonite), magnesia, aluminas, silicas, silica-aluminas, aluminates, titanium oxide, boron oxide, zirconia, aluminum phosphates, titanium phosphates, zirconium phosphates, and carbon. Preferably, said matrix is selected from among the elements of the group that is formed by aluminas, clays, and silicas, in a more preferred manner said matrix is selected from among the aluminas, and in an even more preferred manner said matrix is gamma-alumina.

The catalyst that is used in said stage d) of the process according to the invention is advantageously shaped in the form of grains of different shapes and sizes. It is advantageously used in the form of extrudates that are cylindrical or multilobed, such as bilobed, trilobed, or polylobed of straight or twisted shape, but can optionally be manufactured and used in the form of crushed powder, tablets, rings, balls, wheels, or spheres. Preferably, said catalyst is in the form of extrudates with sizes of between 1 and 10 mm.

Said first oligomerization stage d) of the process according to the invention is performed at a temperature at the reactor inlet of between 40 and 95° C., preferably between 60 and 90° C.; at an absolute pressure of between 0.5 and 10 MPa, preferably between 1 and 8 MPa, and in a preferred manner between 2 and 6 MPa, selected in such a way as to keep products and reagents in liquid form; and at an hourly volumetric flow rate of between 0.1 and 10 h⁻¹ and preferably between 0.4 and 5 h⁻¹.

Said first oligomerization stage d) of the process according to the invention is preferably carried out in a fixed bed. Preferably, said stage is carried out in two fixed-bed reactors in series.

Stage e) for Oligomerization of so-Called “Polynaphtha” Olefins

In accordance with the invention, at least a portion of the effluent from the first oligomerization coming from stage d), the entire effluent coming from the oligomerization stage g), and at least a portion of the light product coming from the fractionation stage 0 are mixed in such a way as to form the feedstock of stage e) for oligomerization of olefins.

This feedstock undergoes a second oligomerization stage e) in the presence of an amorphous catalyst in such a way as to produce a second oligomerization effluent.

The recycling of the entire effluent coming from the oligomerization stage g) and at least a portion of the light product coming from the fractionation stage f) makes it possible to improve the yield of conversion of the olefins having less than 9 carbon atoms by better monitoring of the exothermicity.

The second oligomerization stage e) makes possible the production of an olefin-enriched effluent that has a number of carbon atoms that is greater than or equal to 9.

Said second oligomerization effluent is an olefinic effluent that advantageously comprises less than 50% by weight—and preferably less than 47% by weight—of olefins and paraffins having a number of carbon atoms of between 4 and 9, with the percentages by weight being expressed relative to the total mass of the olefinic and paraffinic C₄-C₉ effluent entering said second oligomerization stage e).

The conversion of isobutene in stage e) is advantageously greater than 80%, preferably greater than 85%, in a preferred manner greater than 90%, and in a very preferred manner greater than 95%.

The conversion of n-butene in stage e) is advantageously greater than 60%, preferably greater than 70%, and in a preferred manner greater than 75%.

The conversion of isopentenes in stage e) is advantageously greater than 80%, preferably greater than 85%, and in a preferred manner greater than 90%.

The conversion of n-pentenes in stage e) is advantageously greater than 55%, preferably greater than 60%, and in a preferred manner greater than 65%.

The conversion of olefins having a number of carbon atoms of between 6 and 9 in stage e) is advantageously greater than 45%, preferably greater than 50%.

The conversion of olefins having a number of carbon atoms that is at least 10 in stage e) is advantageously less than 5%, preferably less than 3%, and in a preferred manner less than 1%.

The rise in temperature in the reactors due to the exothermicity of the reactions in stage e) will be monitored by varying the recycling flow rate of the light product coming from stage f) as well as the fraction of the intermediate product coming from stage f) and sent to stage g).

With the n-butenes, n-pentenes and the olefins having a number of carbon atoms of at least 8 being not very reactive in the second oligomerization stage e), they play the role of heat flywheel in said stage e).

With the paraffins not reacting in the second oligomerization stage e), they play the role of heat flywheel in said stage e).

The catalyst that is used in stage e) of the process according to the invention has the same characteristics as the catalyst that is used in stage d) of the process according to the invention. Preferably, it is identical to the catalyst that is used in stage d) of the process according to the invention.

The catalyst from stage e) can also be a zeolite-based catalyst. In this case, the catalyst preferably comprises at least one zeolite that is selected from the group that consists of the aluminosilicate-type zeolites having an overall Si/Al atomic ratio of greater than 10 and a pore structure of 8, 10 or 12 MR. It preferably consists of a zeolite that is selected from the group that consists of the aluminosilicate-type zeolites having an overall Si/A1 atomic ratio of greater than 10 and a pore structure of 8, 10 or 12 MR. Said zeolite is in a more preferred manner selected from the group that consists of the following zeolites: ferrierite, chabazite, and the zeolites Y and US-Y, ZSM-5, ZSM-12, NU-86, mordenite, ZSM-22, NU-10, ZBM-30, ZSM-11, ZSM-57, ZSM-35, IZM-2, ITQ-6 and IM-5, SAPO, taken by themselves or in a mixture. In a very preferred manner, said zeolite is selected from the group that consists of the zeolites ferrierite, ZSM-5, mordenite, and ZSM-22, taken by themselves or in a mixture. In an even more preferred manner, the zeolite that is used is ZSM-5.

Said second oligomerization stage e) of the process according to the invention is performed at a temperature at the reactor inlet of between 100 and 200° C., preferably between 110 and 160° C.; at an absolute pressure of between 2 and 15 MPa, preferably between 2 and 8 MPa, and in a preferred manner between 3 and 8 MPa; and at an hourly volumetric flow rate of between 0.1 and 10 h⁻¹, and preferably between 0.4 and 5 h⁻¹. In any case, the operating pressure of the process is such that all of the reagents and products are in liquid form in the reaction zone.

Said oligomerization stage e) is advantageously carried out in at least one fixed-bed reactor, preferably at least two fixed-bed reactors in series, and more preferably at least three fixed-bed reactors in series.

The operation of stage e) at an elevated temperature makes possible the production of molecules that are less branched than if this stage were performed at a lower temperature, which leads to obtaining a product whose density is higher.

### Fractionation Stage f)

In accordance with the invention, the second oligomerization effluent coming from stage e) undergoes a fractionation stage f), preferably in at least one distillation column in such a way as to separate said effluent into at least three products respectively corresponding to a light product for the most part comprising the C₂ to C₅ compounds, an intermediate product for the most part comprising the C₆ to C₉ compounds, corresponding to a gasoline fraction, and a middle distillate product for the most part comprising the compounds that have at least 10 carbon atoms whose cutting point is between 150 and 280° C. A bottom product that has an initial boiling point that is higher than 280° C. is also advantageously separated. These cited products are in no way limiting.

Said light product advantageously comprises at least 40% by weight of n-olefins, preferably 50% by weight, and in a preferred manner 55% by weight, with the percentage by weight being expressed relative to the total quantity of butenes present in said light product. The remainder of the light product consists of at least 80% paraffins.

At least one portion of said light product can advantageously be recycled in the second oligomerization stage e) of the process according to the invention.

At least a portion of said light product is advantageously defined as between 0 and 100% by weight of the total mass flow rate of said light product, preferably between 50 and 100% by weight, and more preferably between 75 and 100% by weight.

At least a portion of said intermediate product is treated in an oligomerization stage g).

At least a portion of said intermediate product is advantageously defined as between 80 and 100% of the total mass flow rate of said intermediate product, preferably between 90 and 100%, and more preferably between 95 and 100%.

In accordance with the invention, at least a portion of said middle distillate product is treated in a hydrogenation stage h). A portion of said middle distillate product is defined as at least 80% of the total flow rate of the middle distillate product, preferably at least 90%, and in a preferred manner the entire middle distillate product.

The C16+ compounds will advantageously be separated from said middle distillate before the hydrogenation stage h).

Said stage f) is advantageously carried out with two distillation columns operating in series, with the first distillation column fractionating the effluent coming from the second oligomerization stage e) into a light product for the most part comprising the C₂ to C₅ compounds and a bottom product of the first column, and the second column fractionating said bottom product of the first column into an intermediate product for the most part comprising the C₆ to C₉ compounds and a middle distillate product for the most part consisting of compounds having at least 10 carbon atoms.

Said stage f) can also advantageously be carried out with a column with an inside wall fractionating the effluent coming from the second oligomerization stage e) into a light product for the most part comprising the C₂ to C₅ compounds, an intermediate product for the most part comprising the C₆ to C₉ compounds, and a middle distillate product that for the most part consists of the compounds that have at least 10 carbon atoms.

Said light product advantageously comprises at least 40% by weight of olefins whose number of carbon atoms is less than or equal to 5, preferably at least 50% by weight, and in a preferred manner at least 55% by weight, with this percentage being defined relative to the total weight of said light product.

Said intermediate product advantageously comprises at least 90% by weight of olefins whose number of carbon atoms is between 6 and 9, preferably at least 96% by weight, and more preferably at least 99.8% by weight, with this percentage being defined relative to the total weight of said intermediate product.

Said middle distillate product advantageously comprises at least 90% by weight of olefins whose number of carbon atoms is at least 10, preferably at least 95% by weight, more preferably at least 98% by weight, and in a preferred manner, at least 99.5% by weight, with this percentage being defined relative to the total weight of said middle distillate product.

### Stage g) for Oligomerization of C₈ Olefins Called “Polynaphtha C₈”

In accordance with the invention, at least a portion of said intermediate product that is produced by stage f) of the process according to the invention is treated in a third oligomerization stage g).

The conversion of olefins having 8 carbon atoms in stage g) is advantageously between 20 and 40%. The selectivity of the oligomerization reaction of the olefins having 8 and 9 carbon atoms toward the 140-280° C. fraction is greater than 40%, preferably greater than 45%, and in a preferred manner greater than 50%.

The catalyst that is used in stage g) of the process according to the invention has the same characteristics as the catalyst that is used in stage d) of the process according to the invention. Preferably, it is identical to the catalyst that is used in stage d) of the process according to the invention.

Said oligomerization stage g) of the process according to the invention is performed at a temperature of between 100 and 200° C., preferably between 110 and 160° C.; at an absolute pressure of between 2 and 15 MPa, preferably between 2 and 8 MPa, and in a preferred manner between 3 and 8 MPa; and at an hourly volumetric flow rate of between 0.1 and 5 h⁻¹, and preferably between 0.4 and 2 h⁻¹. In any case, the operating pressure of the process is such that all of the reagents and products are in liquid form in the reaction zone.

Said oligomerization stage g) is advantageously carried out in at least one fixed-bed reactor, preferably at least two fixed-bed reactors in series, and more preferably at least three fixed-bed reactors in series.

The Entire Effluent from Stage g) is Recycled Upstream from Stage e).

### Hydrogenation Stage h)

In accordance with stage h) of the process according to the invention, at least one portion of said middle distillate product coming from stage f) undergoes a stage for hydrogenation of olefins into paraffins to make them able to be incorporated into the fuel pool.

Preferably, at least a portion—and preferably all—of said middle distillate product coming from stage f) is brought into contact with a hydrogen-rich gas in the presence of a catalyst that comprises at least one metal of group VIII, advantageously selected from among palladium and nickel, taken by itself or in a mixture, and a substrate that is advantageously selected from among alumina, silica or silica-alumina.

The catalyst that is used in said hydrogenation stage h) comprises a palladium content of between 0.1 and 5% by weight and/or a nickel oxide content that is advantageously between 15 and 40% by weight relative to the total mass of the catalyst. This nickel can be promoted with molybdenum or can also be partially sulfurized. When the metal content of the catalyst increases, the operating temperature of the reaction section is to be adapted to the decrease for compensating for the increase in catalytic activity.

The hydrogenation stage h) is performed at a temperature of between 100 and 350° C. at the reactor inlet and at a pressure of between 2 and 4 MPa and at an hourly volumetric flow rate of between 1 and 5 h⁻¹. The molar ratio of hydrogen to hydrocarbons H₂/HC is between 10 and 450.

The unreacted hydrogen is separated leaving the reactor so as to be recycled as input. A fraction of the total flow of the reaction effluent from which hydrogen is removed is also recycled as input in such a way as to be used as a heat flywheel in the reaction. The flow rate of said recycled fraction represents between 2 and 5 times the mass flow rate of said portion of said middle distillate product coming from stage 0 entering stage h), i.e., before the recycling of hydrogen and said fraction.

The reactors that are used in stage h) are multi-bed reactors with a recycling of a portion of the reaction effluent for diluting the feedstock and controlling the exotherm. The hydrogenation almost exclusively concerns olefins; the reaction is therefore very exothermic (between 100 and 300° C. of exotherm).

The performance of the hydrogenation is validated by measuring the smoke point and current gums that will advantageously be greater than 25 mm for the smoke point and less than 7 mg/100 ml for the current gum content. This is generally reflected by measuring the bromine number in accordance with the standard ASTM D2710, which is advantageously at most 10 mg of Br/100 g when these limits for the smoke point and the current gums are observed.

The effluent coming from the optional hydrogenation stage for the most part contains hydrocarbons that can be upgraded and incorporated into the kerosene and/or diesel fuel pool, and preferably kerosene.

The hydrocarbon yield of which the number of carbon atoms is at least 10 of said hydrogenation stage h) is greater than 90%, preferably greater than 95%. The olefin content in the effluent from said stage h) is between 0 and 5% of the total weight of said effluent, preferably between 0 and 2% by weight.

An optional separation stage following the hydrogenation stage h) is advantageously carried out for making possible the fractionation into a kerosene fraction and/or a diesel fuel fraction and/or a fraction having a boiling point that is higher than 360° C. and/or light fractions.

### EXAMPLES

Examples 1-3 and 5-6 illustrate the invention. Example 4 illustrates the prior art.

### Example 1

### Compliant

This example illustrates the invention.

### Description of the Feedstock Comprising Butanol and Pentanol

The feedstock that comprises butanol and pentanol used in the example was treated by a series of stages for distillation and running over molecular sieves. Following these treatments, the purified feedstock has the composition that is indicated in Table 6, “stage a) feedstock” column. The purified feedstock has an ionic impurity content of less than 100 ppm, a metal content of less than 50 ppm, an oxidized impurity content of less than 150 ppm, and a total content of nitrogen-containing and sulfur-containing compounds of less than 30 ppm.

### Stage a): Dehydration of the Purified Feedstock

The purified feedstock undergoes an isomerizing dehydration stage a). Said stage a) is performed at a temperature of 400° C., at a pressure of 0.85 MPa at the reactor inlet, and at an hourly volumetric flow rate of 5 h⁻¹ in the presence of a ZSM-23 catalyst in such a way as to maximize the production of butenes and pentenes.

The effluent from stage a), for the most part olefinic, has the composition that is indicated in Table 6, “stage a) effluent” column. The n-butanol is converted at a level of 99.8% into a mixture of butenes and water comprising 24.8% by mass of butenes. The pentan-1-ol is converted at a level of 99.5% into a mixture of pentenes with 25% of the thermodynamic equilibrium and water. The reaction also produces a certain number of co-products: oxidized elements and light olefins.

Stage b): Separation of Water from the Olefinic Effluent

The effluent that is for the most part olefinic and that comes from stage a) is next directed to a decanting flask to carry out the separation of water there. The separation is carried out at 50° C. and 0.75 MPa in such a way as to promote the liquid-liquid segregation and therefore the separation of hydrocarbons from water. The residual content of water in the organic liquid effluent is then 1,800 ppm.

The fraction of oxidized elements, nitrogen-containing elements, and water in the organic liquid effluent coming from stage b) is 0.4%.

### Stage c): Purification of the Organic Liquid Effluent

The organic liquid effluent coming from stage b) next passes through a purification stage so as to remove the major portion of the remaining water and the compounds that can interfere with the downstream catalytic beds. This purification stage makes it possible to remove partially the sulfur-containing, nitrogen-containing, and oxidized elements remaining in a molecular sieve 13x, and then to remove partially the remaining water in a molecular sieve 3A.

The composition of the purified organic effluent coming from stage c) is indicated in Table 1:

TABLE 1

| | Composition (% by Weight) |
|---|---|
| Water | 300 ppm |
| Nitrogen-Containing, Sulfur- | <1 ppm |
| Containing, and Inorganic | |
| Elements | |
| Oxidized Compounds | 0.05% |

### Stage d): Stage for Selective Oligomerization of Isobutene and Isopentenes

The purified organic effluent coming from stage c) has the composition that is indicated in Table 6, “stage d) feedstock” column. It is treated in the first oligomerization stage d), which is performed in the presence of the catalyst IP811 that is marketed by Axens. The catalyst IP811 is an amorphous silica-alumina catalyst. The operating conditions of stage d) are a temperature of 60° C., a pressure of 3 MPa at the reactor inlet, and an hourly volumetric flow rate in the reactors of 2 h⁻¹ in such a way as to promote very heavily the oligomerization of isobutene and isopentenes while limiting the reaction of n-butenes and n-pentenes.

The effluent from the oligomerization stage d) has the composition indicated in Table 6, “stage e) feedstock” column.

At the end of stage d), from 1 kg of a mixture comprising butanol and pentanol introduced into stage a), 234 g of olefins having a number of carbon atoms that is greater than or equal to 6 is produced.

### Stage e): Stage for Oligomerization of Olefins

The effluent from the selective oligomerization stage d) is mixed with 75% of the total flow of effluent from the top of the fractionation stage f) as well as all of the effluent from the oligomerization stage g) of the C₈-C₉ olefins. This mixture has the composition indicated in Table 7, “stage e) feedstock” column. It is next treated in stage e). The latter is performed in the presence of the catalyst IP811 that is marketed by Axens. The operating conditions of stage e) are a temperature of 110° C., a pressure of 6 MPa at the reactor inlet, and an hourly volumetric flow rate in the reactors of 2 h⁻¹.

The effluent from the oligomerization stage e) has the composition that is indicated in Table 6, “stage f) feedstock” column.

### Stage f): Fractionation Stage of the Oligomerization Effluents

The effluent from stage e) next undergoes a fractionation stage f) in such a way as to separate a light product that comprises the C₂ to C₅ compounds, an intermediate product comprising the C₆ to C₉ compounds, and a middle distillate product, which will constitute in part the kerosene fraction, compound of C₁₀⁺. The distribution of the separated products is presented in Table 2.

TABLE 2
Distribution of the Products at the End of Stage f)

| Product | Composition (% by Weight) |
|---|---|
| Light Product | 24.9% |
| Intermediate Product | 22.5% |
| Middle Distillate Product | 52.6% |

From 1 kg/h of a mixture comprising butanol and pentanol entering stage a), the following are obtained: 328 g/h of a light product, 296 g/h of an intermediate product, and 690 g/h of a middle distillate product leaving stage f). A large portion of the first two products is recycled to obtain these results: 75% of the light product is recycled entering stage e), and 100% of the intermediate product is directed toward stage g) so as to oligomerize the C₅-C₉ olefins before being redirected toward stage e).

The light product contains 500 ppm by mass of carbon-containing compounds other than C₁-C₅. The intermediate product contains 2,000 ppm by mass of carbon-containing compounds other than C₆-C₉. The middle distillate product contains 0.5% by mass of C₁-C₉ carbon-containing compounds.

### Stage g): Oligomerization of the C8 Olefins

100% of the intermediate product coming from stage f) is directed toward stage g) for oligomerization of the C₈ and C₉ olefins. This stage is performed in the presence of the catalyst IP811 that is marketed by Axens. The operating conditions of stage g) are a temperature of 110° C., a pressure of 6 MPa at the reactor inlet, and an hourly volumetric flow rate in the reactors of 2 h⁻¹.

The composition of the effluent leaving stage g) is presented in Table 3. It is next recycled entering stage d).

TABLE 3

| Distribution by Mass of the | |
|---|---|
| Hydrocarbon-Containing | |
| Compounds | % by Weight |
| Paraffins | 0.7% |
| 90° C.-140° C. | 65.0% |
| 140° C.-280° C. | 27.3% |
| 280° C.+ | 7% |

### Stage h): Hydrogenation of Olefins

The middle distillate product coming from the fractionation stage f) is directed toward stage h) for hydrogenation of olefins that is performed with a catalyst LD746 that is marketed by Axens and in which the major portion of the olefins will be hydrogenated while minimizing the production of light hydrocarbon-containing molecules. This stage is performed at a temperature of 160° C. and a pressure of 2.5 MPa at the reactor inlet. The hourly volumetric flow rate in the reaction section is 3 h⁻¹, and the H₂/HC ratio is equal to 100.

Stage h) produces a mixture of alkanes for the most part having between 9 and 16 carbon atoms whose distribution is presented in Table 4.

TABLE 4

| Composition by Mass of | |
|---|---|
| the Effluent from Stage h) | |
| (H₂ Not Included) | % by Weight |
| 140° C.-280° C. | 74.2 |
| C₁-C₅ | 1.1 |
| 90° C.-140° C. | 1.7 |
| 280° C.+ | 23 |

The effluent from the hydrogenation stage h) is next fractionated, and then the corresponding fractions are mixed with the non-recycled fractions of light and intermediate products obtained from stage f).

The distribution of the products coming from the process that is performed according to Example 1 is presented in Table 5.

TABLE 5

| | % by Weight |
|---|---|
| Kerosene Fraction | 66.4% |
| Lights (C₁-C₅) | 11.5% |
| 90° C.-140° C. | 1.5% |
| 280° C.+ | 20.6% |

Leaving stage h), from 1 kg/h of a mixture comprising butanol and pentanol entering stage a), 517 g/h of bio-kerosene is obtained.

TABLE 6
Primary Streams of Example 1

| | Feedstock | Effluent | Feedstock | Effluent | Feedstock | Feedstock | Feedstock | Feedstock |
|---|---|---|---|---|---|---|---|---|
| | from Stage | from Stage | from Stage | from Stage | from Stage | from Stage | from Stage | from Stage |
| Description | a) | a) | d) | d) | e) | f) | g) | h) |
| Temperature | 400 | 5 | 60 | 110 | 110 | 150 | 110 | 160 |
| (° C.) | | | | | | | | |
| Pressure | 0.85 | 0.75 | 3.0 | 6.0 | 6 | 5.9 | 6.0 | 2.5 |
| (MPa) | | | | | | | | |
| Mass Flow | 34.7 | 34.7 | 18.7 | 18.7 | 31.7 | 31.7 | 7.1 | 16.8 |
| Rate (t/h) | | | | | | | | |
| Composition | | | | | | | | |
| (% by | | | | | | | | |
| Weight) of | | | | | | | | |
| n-Butanol | 34.5% | 0.1% | | | | | | |
| Pentan-1-ol | 34.5% | 0.2% | | | | | | |
| Paraffins | | 0.3% | 0.6% | 1.3% | 8.9% | 10.7% | | |
| C3= and | | 0.4% | 0.7% | 0.7% | 0.4% | | | |
| Diolefins | | | | | | | | |
| i-C4= | | 6.6% | 12.2% | 0.2% | 0.1% | | | |
| n-C4= | | 18.2% | 33.8% | 31.8% | 22.6% | 5.2% | 0.2% | |
| i-C5= | | 6.6% | 12.3% | 2.2% | 1.4% | 0.1% | | |
| n-C5= | | 19.6% | 36.4% | 33.5% | 26.4% | 9% | | |
| 90-140° C. | | 2.1% | 4% | 13.1% | 22.3% | 22.5% | 99.8 | |
| 140-280° C. | | | | 15.5% | 15.2% | 40.2% | | 76% |
| 280+° C. | | | | 1.7% | 2.6% | 12.4% | | 23% |
| H2 | | | | | | | | 1% |
| H2O | 30% | 45.9% | | | | | | |
| Other | 1% | | | | | | | |
| Alcohols | | | | | | | | |
| Aldehydes | | 0.1% | | | | | | |

### Example 2

### Case of Operating with a Low Content of C4

In this example, also in accordance with the invention, the alcohol feedstock entering the dehydration stage a) consists of 10% butanol and 90% pentanol.

### Description of the Feedstock Comprising Butanol and Pentanol

The feedstock that comprises the butanol and pentanol used in the example has been treated by a series of stages of distillation and running over molecular sieves. Following these treatments, the purified feedstock has the following composition:

| | Composition by Mass |
|---|---|
| n-Butanol | 7% |
| Pentan-1-ol | 63% |
| Water | 30% |

The feedstock also comprises a content of ionic impurities of less than 100 ppm, a metal content of less than 50 ppm, a content of oxidized impurities of less than 150 ppm, and a total content of nitrogen-containing and sulfur-containing compounds of less than 30 ppm.

### Stage a): Dehydration of the Purified Feedstock

The operating conditions are identical to stage a) of Example 1. The composition of the effluent from the dehydration stage a) is described in Table 11.

Stage b): Separation of Water from the Olefinic Effluent

Stage b) for separation of water takes place under the same conditions as stage b) of Example 1. The composition of the effluent from stage b) for separation from water is described in Table 11.

### Stage c): Purification of the Organic Liquid Effluent

Stage c) for purification of the organic liquid effluent takes place under the same conditions as stage c) of Example 1.

### Stage d): Stage for Selective Oligomerization of Isobutene and Isopentenes

The first oligomerization stage d) takes place under the same conditions as stage d) of Example 1. The composition of the effluent leaving the reaction is presented in Table 11.

At the end of this stage, from 1 kg/h of a mixture comprising butanol and pentanol entering stage a), 229 g/h of olefins having more than 6 carbon atoms is produced.

The effluent from stage d) is next directed toward stage e) for oligomerization of olefins.

### Stage e): Stage for Oligomerization of Olefins

Stage e) is performed under the same conditions and with the same catalyst as Example 1. The composition of the second oligomerization effluent is indicated in Table 11.

### Stage f): Stage for Fractionation of the Oligomerization Effluents

The effluent from stage e) is next fractionated into 3 products in stage f), in the same manner as in Example 1. The distribution of the separated products is presented in Table 7.

TABLE 7

| Product | % by Weight |
|---|---|
| Light Product | 30.1% |
| Intermediate Product | 11.6% |
| Middle Distillate Product | 58.3% |

From 1 kg/h of a mixture comprising butanol and pentanol entering stage a), there is obtained: 359 g/h of a light product, 138 g/h of an intermediate product, and 695 g/h of a middle distillate product. 75% by mass of the light product is recycled entering stage e), and 100% by mass of the intermediate product is directed toward stage g).

The light product contains 500 ppm by mass of carbon-containing compounds other than C₁-C₄. The intermediate product contains 2,000 ppm by mass of carbon-containing compounds other than C₅-C₉. The middle distillate product contains 0.5% by mass of C₁-C₉ carbon-containing compounds.

### Stage g): Oligomerization of the C8-C9 Olefins

Stage g) is performed under the same conditions and with the same catalyst as stage g) of Example 1. The composition of the oligomerization effluent of the C8-C9 olefins is indicated in Table 8.

TABLE 8
Effluent from Stage g)

| Distribution by Mass of the | |
|---|---|
| Hydrocarbon-Containing | |
| Compounds | % by Weight |
| Paraffins | 0.7% |
| 90° C.-140° C. | 65.0% |
| 140° C.-280° C. | 27.3% |
| 280° C.+ | 7% |

### Stage h): Hydrogenation of Olefins

The middle distillate product coming from the fractionation stage f) is directed toward stage h) for hydrogenation of olefins. Stage h) is performed under the same conditions and with the same catalyst as Example 1. The hydrogenation effluent has the composition presented in Table 9.

TABLE 9

| Composition by Mass of the | |
|---|---|
| Effluent from Stage h) | |
| (H₂ Not Included) | % by Weight |
| 140° C.-280° C. | 76.9 |
| C₁-C₅ | 1.1 |
| 90° C.-140° C. | 1.7 |
| 280° C.+ | 20.3 |

The effluent from the hydrogenation stage h) is next fractionated, and then the corresponding fractions are mixed with the non-recycled fractions of light and intermediate products obtained from stage f).

The distribution of the products coming from the process that is performed according to Example 2 is presented in Table 10.

TABLE 10

| | % by Weight |
|---|---|
| Kerosene Fraction | 68.2% |
| Lights (C₁-C₅) | 12.3% |
| 90° C.-140° C. | 1.5% |
| 280° C.+ | 18.0% |

Leaving stage h), from 1 kg/h of a mixture comprising butanol and pentanol entering stage a), 541 g/h of bio-kerosene is obtained.

TABLE 11
Primary Streams of Example 2

| | Feedstock | Effluent | Feedstock | Effluent | Feedstock | Feedstock | Feedstock | Feedstock |
|---|---|---|---|---|---|---|---|---|
| | from Stage | from Stage | from Stage | from Stage | from Stage | from Stage | from Stage | from Stage |
| Description | a) | a) | d) | d) | e) | f) | g) | h) |
| Temperature | 400 | 55 | 60 | 110 | 110 | 150 | 110 | 160 |
| (° C.) | | | | | | | | |
| Pressure | 0.85 | 0.75 | 3.0 | 6.0 | 6 | 5.9 | 6.0 | 2.5 |
| (MPa) | | | | | | | | |
| Mass Flow | 33.5 | 33.5 | 18.2 | 18.2 | 27.6 | 27.6 | 3.2 | 16.2 |
| Rate (t/h) | | | | | | | | |
| Composition | | | | | | | | |
| (% by | | | | | | | | |
| Weight) of | | | | | | | | |
| n-Butanol | 7% | 0.% | | | | | | |
| Pentan-1-ol | 63% | 0.3% | | | | | | |
| Paraffins | | 0.4% | 0.8% | 1.4% | 9.2% | 11.0% | | |
| C3= and | | 0.1% | 0.1% | 0.1% | 0.1% | | | |
| Diolefins | | | | | | | | |
| i-C4= | | 1.3% | 2.4% | | | | 0.2% | |
| n-C4= | | 3.7% | 6.6% | 6.2% | 5.0% | 1.1% | | |
| i-C5= | | 12% | 21.7% | 3.9% | 2.7% | 0.2% | | |
| n-C5= | | 35.4% | 64.3% | 59.2% | 52.4% | 17.8% | | |
| 90-140° C. | | 2.2% | 4% | 7.2% | 12.3% | 11.6% | 99.8 | |
| 140-280° C. | | | | 19.9% | 16.3% | 46.2% | | 79% |
| 280+° C. | | | | 2% | 2.1% | 12.1% | | 20% |
| H2 | | | | | | | | 1% |
| H2O | 29% | 44.6% | | | | | | |
| Other | 1% | | | | | | | |
| Alcohols | | | | | | | | |
| Aldehydes | | 0.1% | | | | | | |

### Example 3

### Case of Operating with a Low Content of C5

In this example, in accordance with the invention, the alcohol feedstock entering the dehydration stage a) consists of 90% butanol and 10% pentanol.

### Description of the Feedstock Comprising Butanol and Pentanol

The feedstock that comprises the butanol and pentanol that are used in the example has been treated by a series of stages of distillation and running over molecular sieves. Following these treatments, the purified feedstock has the following composition:

| | Composition by Mass |
|---|---|
| n-Butanol | 63% |
| Pentan-1-ol | 7% |
| Water | 30% |

The feedstock also comprises a content of ionic impurities of less than 100 ppm, a metal content of less than 50 ppm, a content of oxidized impurities of less than 150 ppm, and a total content of nitrogen-containing and sulfur-containing compounds of less than 30 ppm.

### Stage a): Dehydration of the Purified Feedstock

The operating conditions are identical to stage a) of Example 1.

The composition of the effluent from the dehydration stage a) is described in Table 16.

Stage b): Separation of Water from the Olefinic Effluent

Stage b) for separation of water takes place under the same conditions as stage b) of Example 1. The composition of the effluent from the dehydration stage a) is described in Table 16.

### Stage c): Purification of the Organic Liquid Effluent

Stage c) for purification of the organic liquid effluent takes place under the same conditions as stage c) of Example 1.

### Stage d): Stage for Selective Oligomerization of Isobutene and Isopentenes

First oligomerization stage d) takes place under the same conditions as stage d) of Example 1. The composition of the effluent leaving the reaction is presented in Table 16.

At the end of this stage, from 1 kg/h of a mixture comprising butanol and pentanol entering stage a), 239 g/h of olefins having more than 6 carbon atoms is produced.

The effluent from stage d) is next directed toward stage e) for oligomerization of olefins.

### Stage e): Stage for Oligomerization of Olefins

Stage e) is performed under the same conditions and with the same catalyst as Example 1. The composition of the second oligomerization effluent is indicated in Table 16.

### Stage f): Stage for Fractionation of the Oligomerization Effluents

The effluent from stage e) is next fractionated into 3 products in stage f), in the same manner as in Example 1. The distribution of the separated products is presented in Table 12.

TABLE 12

| Product | % by Weight |
|---|---|
| Light Product | 21.7% |
| Intermediate Product | 26.6% |
| Middle Distillate Product | 51.7% |

From 1 kg/h of a mixture comprising butanol and pentanol entering stage a), there is obtained: 286 g/h of a light product, 351 g/h of an intermediate product, and 682 g/h of a middle distillate product. 75% by mass of the light product is recycled entering stage e), and 100% by mass of the intermediate product is directed toward stage g).

The light product contains 500 ppm by mass of carbon-containing compounds other than C₁-C₄. The intermediate product contains 2,000 ppm by mass of carbon-containing compounds other than C₅-C₉. The middle distillate product contains 0.5% by mass of C₁-C₉ carbon-containing compounds.

### Stage g): Oligomerization of the C8-C9 Olefins

Stage g) is performed under the same conditions and with the same catalyst as stage g) of Example 1. The composition of the oligomerization effluent of the C8-C9 olefins is indicated in Table 13.

TABLE 13
Effluent from Stage g)

| Distribution by Mass of the | |
|---|---|
| Hydrocarbon-Containing | |
| Compounds | % by Weight |
| Paraffins | 0.7% |
| 90° C.-140° C. | 65.7% |
| 140° C.-280° C. | 32.9% |
| 280° C.+ | 0.7% |

### Stage h): Hydrogenation of Olefins

The middle distillate product coming from the fractionation stage f) is directed toward stage h) for hydrogenation of olefins. Stage h) is performed under the same conditions and with the same catalyst as Example 1. The hydrogenation effluent has the composition that is presented in Table 14.

TABLE 14

| Composition by Mass of the | |
|---|---|
| Effluent from Stage h) | |
| (H₂ Not Included) | % by Weight |
| 140° C.-280° C. | 76.3 |
| C₁-C₅ | 1.1 |
| 90° C.-140° C. | 1.7 |
| 280° C.+ | 21 |

The effluent from the hydrogenation stage h) is next fractionated, and then the corresponding fractions are mixed with the non-recycled fractions of light and intermediate products obtained from stage f).

The distribution of the products coming from the process that is performed according to Example 3 is presented in Table 15.

TABLE 15

| | % by Weight |
|---|---|
| Kerosene Fraction | 69.0% |
| Lights (C₁-C₅) | 10.4% |
| 90° C.-140° C. | 1.5% |
| 280° C.+ | 19.0% |

Leaving stage h), from 1 kg/h of a mixture comprising butanol and pentanol entering stage a), 525 g/h of bio-kerosene is obtained.

TABLE 16
Primary Streams of Example 3

| | Feedstock | Effluent | Feedstock | Effluent | Feedstock | Feedstock | Feedstock | Feedstock |
|---|---|---|---|---|---|---|---|---|
| | from Stage | from Stage | from Stage | from Stage | from Stage | from Stage | from Stage | from Stage |
| Description | a) | a) | d) | d) | e) | f) | g) | h) |
| Temperature | 400 | 55 | 60 | 110 | 110 | 150 | 110 | 160 |
| (° C.) | | | | | | | | |
| Pressure | 0.85 | 0.75 | 3.0 | 6.0 | 6 | 5.9 | 6.0 | 2.5 |
| (MPa) | | | | | | | | |
| Mass Flow | 34 | 34 | 18.0 | 18.0 | 31.4 | 31.4 | 8.4 | 16.4 |
| Rate (t/h) | | | | | | | | |
| Composition | | | | | | | | |
| (% by | | | | | | | | |
| Weight) of | | | | | | | | |
| n-Butanol | 63% | 0.1% | | | | | | |
| Pentan-1-ol | 7% | 0% | | | | | | |
| Paraffins | | 0.2% | 0.4% | 1.1% | 8.7% | 10.6% | | |
| C3= and | | 0.7% | 1.3% | 1.3% | 0.8% | | | |
| Diolefins | | | | | | | | |
| i-C4= | | 11.9% | 22.4% | 0.4% | 0.3% | | 0.2% | |
| n-C4= | | 32.9% | 62% | 58.3% | 40.5% | 9.3% | | |
| i-C5= | | 1.3% | 2.5% | 0.5% | 0.3% | | | |
| n-C5= | | 3.9% | 7.4% | 6.8% | 5.3% | 1.8% | | |
| 90-140° C. | | 2.1% | 4% | 19.2% | 28.3% | 26.6% | 99.8 | |
| 140-280° C. | | | | 10.9% | 14.9% | 40.6% | | 78% |
| 280+° C. | | | | 1.5% | 1.0% | 11.1% | | 21% |
| H2 | | | | | | | | 1% |
| H2O | 29% | 46.8% | | | | | | |
| Other | 1% | | | | | | | |
| Alcohols | | | | | | | | |
| Aldehydes | | 0.1% | | | | | | |

### Example 4 (Non-Compliant)

### Results According to the Oligomerization Diagram Presented in the Patent FR 2975103

In this example, there is no dehydration stage, and oligomerization is carried out in two successive stages, without recycling a portion of the product for monitoring the exotherm and without oligomerization of the C8 and C9.

### Description of the Butenes/Pentenes/Hexenes Feedstock

The feedstock consists of a mixture of the LCN fraction and a C4 fraction coming from an FCC unit. It has the following composition:

| | Composition by Mass |
|---|---|
| n-Butenes | 16.1% |
| i-Butenes | 5.7% |
| Butanes | 13.9% |
| i-Pentenes | 16.7% |
| n-Pentenes | 15.6% |
| Pentanes | 17.7% |
| Hexenes | 7.2% |
| Hexanes | 4.6% |

Its density is 0.6312. It also contains organic nitrogen-containing compounds totaling a content of 7.6 ppm by weight expressed in terms of elementary nitrogen and 14 ppm of elementary sulfur.

### Stage c): Purification of the Organic Liquid Effluent

The olefinic feedstock next passes through a purification stage so as to remove the major portion of the remaining water and the compounds that can interfere with the downstream catalytic beds. This purification stage is carried out at 50 bar absolute and at ambient temperature. The content of nitrogen-containing compounds leaving this stage is 0.5 ppm by weight.

### Stage d): Stage for Selective Oligomerization of Isobutene and Isopentenes

The purified organic effluent coming from stage c) is treated in the first oligomerization stage d), which is performed in the presence of the catalyst IP811 that is marketed by Axens. The catalyst IP811 is an amorphous silica-alumina catalyst. The operating conditions of stage d) are a temperature of between 60° C. and 110° C. in such a way as to keep the conversion of C5 iso-olefins constant between 80% and 85%.

Under these conditions, the conversion of the isobutene is greater than 95%. A mean increase of 2° C. every 100 hours is necessary for maintaining this conversion. Under these conditions, the conversion of the normal C4 and C5 olefins is between 10 and 20%.

### Stage e): Stage for Oligomerization of Olefins

The effluent from the first oligomerization stage d) is sent integrally into the third bed that carries out the second oligomerization stage, in which the temperature is raised gradually between 150° C. and 230° C., so as to keep the conversion of C5 olefins constant between 75% and 80%. The conversion of the C4 olefins is greater than 80%. A mean increase of 1° C./100 h is necessary for maintaining this conversion.

The effluent from the third stage is separated by distillation into four fractions:
A C4-fraction (16% by weight)
A 15° C.-140° C. fraction (42% by weight), constituting the light gasoline fraction, whose research octane number (RON) is 95.5.
A 140° C.-280° C. fraction (32% by weight), which is next hydrogenated in the hydrogenation stage h) (conditions that are identical to those described in the preceding examples) for providing a kerosene fraction, whose characteristics are as follows:
   Smoke point: 38° C.; flash point: 48° C.; Crystallization point: <−65° C.; Density: 0.778
A 280⁺ residue (10% by weight).

### Stage h): Hydrogenation of Olefins

The entire 140° C.-280° C. fraction obtained from stage e) for oligomerization of olefins is next hydrogenated in the hydrogenation stage h) (conditions identical to those described in the preceding examples) for providing a kerosene fraction, of which the characteristics are the following:
Smoke point: 38° C.; flash point: 48° C.; Crystallization point: <−65° C.;
Density: 0.778

From 1 kg/h of input olefinic feedstock, 320 g/h of kerosene is produced as output.

### Example 5

### Additions of Aromatic Compounds and the Effect on the Density

The kerosene product that is obtained in Example 1 conforms to a density of approximately 780 kg/m³. Mesitylene (1,3,5-trimethylbenzene), whose density is 865 kg/m³, is added to this product.

The addition of mesitylene to our kerosene up to a content of 8% by volume raises the density of the mixture to 787 kg/m3.

The addition of mesitylene to our kerosene up to a content of 25% by volume raises the density to 801 kg/m3.

It therefore always remains within the density limits set by the standard ASTM D7566-11a, even when adding aromatic compounds up to the limits provided by said standard.

### Example 6

### Compliant

This example illustrates the invention with a zeolitic catalyst in stage e).

### Description of the Feedstock Comprising Butanol and Pentanol

The feedstock comprising butanol and pentanol used in the example is identical to the one used in Example 1. It has also been treated by a series of stages for distillation and running over molecular sieves, and it has an impurity content that is identical to the one of Example 1. Its composition is described in Table 22.

### Stage a): Dehydrogenation of the Purified Feedstock

The isomerizing dehydration stage is identical to stage a) of Example 1.

The composition of the effluent of the dehydration stage a) is the same as the one of the effluent of stage a) of Example 1. It is described in Table 6.

Stage b): Separation of Water from the Olefinic Effluent

Stage b) for separation of water from the olefinic effluent is identical to the one of stage b) of Example 1.

The composition of the effluent from stage b) for separation of water is described in Table 22.

### Stage c): Purification of the Organic Liquid Effluent

Stage c) for purification of the organic liquid effluent takes place under the same conditions as stage c) of Example 1. The composition of the purified organic effluent of stage c) is provided in Table 17.

TABLE 17

| | Composition | |
|---|---|---|
| | (% by Weight) | |
| Water | 300 | ppm |
| Nitrogen-Containing, Sulfur- | <1 | ppm |
| Containing, and Inorganic | | |
| Elements | | |

| | |
|---|---|
| Oxidized Compounds | 0.05% |

### Stage d): Stage for Selective Oligomerization of Isobutene and Isopentenes

The first oligomerization stage d) takes place under the same conditions as stage d) from Example 1. The composition of the effluent leaving the reaction is presented in Table 22.

At the end of stage d), from 1 kg of a mixture comprising butanol and pentanol introduced into stage a), 234 g of olefins having a number of carbon atoms that is greater than or equal to 6 is produced.

The effluent from stage d) is next directed toward stage e) for oligomerization of olefins.

At the end of stage d), from 1 kg of a mixture comprising butanol and pentanol introduced into stage a), 234 g of olefins having a number of carbon atoms that is greater than or equal to 6 is produced.

### Stage e): Stage for Oligomerization of Olefins

The effluent from the selective oligomerization stage d) is mixed with 75% of the total flow of effluent from the top of the fractionation stage f) as well as all of the effluent from the oligomerization stage g) of the C₈-C₉ olefins. This mixture has the composition indicated in Table 7, “stage e) feedstock” column. It is next treated in stage e). The latter is performed in the presence of a zeolitic catalyst ZSM5 that is prepared by mixing the ZSM-5 zeolite with an SB3-type alumina that is provided by the Condea Company. The operating conditions of stage e) are a temperature of 230° C., a pressure of 5 MPa at the reactor inlet, and an hourly volumetric flow rate in the reactors of 2 h⁻¹.

The effluent from the oligomerization stage e) has the composition that is indicated in Table 22, “stage f) feedstock” column.

### Stage f): Fractionation Stage of the Oligomerization Effluents

The effluent from stage e) is next fractionated into 3 products in stage f) in the same manner as in Example 1. The distribution of the separated products is presented in Table 18.

TABLE 18

| Product | % by Weight |
|---|---|
| Light Product | 21.7% |
| Intermediate Product | 21.8% |
| Middle Distillate Product | 56.5% |

From 1 kg/h of a mixture comprising butanol and pentanol entering stage a), the following are obtained: 271 g/h of a light product, 272 g/h of an intermediate product, and 704 g/h of a middle distillate product. 75% by mass of the light product is recycled entering stage e), and 100% by mass of the intermediate product is directed toward stage g).

The light product contains 500 ppm by mass of carbon-containing compounds other than C₁-C₄. The intermediate product contains 2,000 ppm by mass of carbon-containing compounds other than C₅-C₉. The middle distillate product contains 0.5% by mass of C₁-C₉ carbon-containing compounds.

### Stage g): Oligomerization of the C8 Olefins

Stage g) is performed under the same conditions and with the same catalyst as stage g) of Example 1. The composition of the effluent for oligomerization of the C8-C9 olefins is indicated in Table 19.

TABLE 19
Effluent from Stage g)

| Distribution by Mass of the | |
|---|---|
| Hydrocarbon-Containing | |
| Compounds | % by Weight |
| Paraffins | 0.7% |
| 90° C.-140° C. | 65.0% |
| 140° C.-280° C. | 27.3% |
| 280° C.+ | 7% |

### Stage h): Hydrogenation of Olefins

The middle distillate product coming from the fractionation stage f) is directed toward stage h) for hydrogenation of olefins. Stage h) is performed under the same conditions and with the same catalyst as Example 1. The hydrogenation effluent has the composition that is presented in Table 20.

TABLE 20

| Composition by Mass of the | |
|---|---|
| Effluent from Stage h) | |
| (H₂ Not Included) | % by Weight |
| 140° C.-280° C. | 70.1 |
| C₁-C₅ | 1.1 |
| 90° C.-140° C. | 1.7 |
| 280° C.+ | 27.2 |

The effluent from the hydrogenation stage h) is next fractionated, and then the corresponding fractions are mixed with the non-recycled fractions of light and intermediate products obtained from stage f).

The distribution of the products coming from the process that is performed according to Example 2 is presented in Table 21.

TABLE 21

| | % by Weight |
|---|---|
| Kerosene Fraction | 64.0% |
| Lights (C₁-C₅) | 9.6% |
| 90° C.-140° C. | 1.5% |
| 280° C.+ | 24.8% |

Leaving stage h), from 1 kg/h of a mixture comprising butanol and pentanol entering stage a), 499 g of bio-kerosene is obtained.

TABLE 22
Primary Streams of Example 6

| | Feedstock | Effluent | Feedstock | Effluent | Feedstock | Feedstock | Feedstock | Feedstock |
|---|---|---|---|---|---|---|---|---|
| | from Stage | from Stage | from Stage | from Stage | from Stage | from Stage | from Stage | from Stage |
| Description | a) | a) | d) | d) | e) | f) | g) | h) |
| Temperature | 400 | 55 | 60 | 110 | 110 | 150 | 110 | 160 |
| (° C.) | | | | | | | | |
| Pressure | 0.85 | 0.75 | 3.0 | 6.0 | 6 | 5.9 | 6.0 | 2.5 |
| (MPa) | | | | | | | | |
| Mass Flow | 35.9 | 34.7 | 19.4 | 19.4 | 31.2 | 31.2 | 6.8 | 17.8 |
| Rate (t/h) | | | | | | | | |
| Composition | | | | | | | | |
| (% by | | | | | | | | |
| Weight) of | | | | | | | | |
| n-Butanol | 34.5% | 0.1% | | | | | | |
| Pentan-1-ol | 34.5% | 0.2% | | | | | | |
| Paraffins | | 0.3% | 0.6% | 1.3% | 10.9% | 13.3% | | |
| C3= and | | 0.4% | 0.7% | 0.7% | 0.4% | | | |
| Diolefins | | | | | | | | |
| i-C4= | | 6.6% | 12.2% | 0.2% | 0.2% | | 0.2% | |
| n-C4= | | 18.2% | 33.8% | 31.8% | 21.1% | 1.9% | | |
| i-C5= | | 6.6% | 12.3% | 2.2% | 1.6% | 0.1% | | |
| n-C5= | | 19.6% | 36.4% | 33.5% | 28.4% | 6.4% | | |
| 90-140° C. | | 2.1% | 4% | 13.1% | 25.2% | 21.8% | 99.8 | |
| 140-280° C. | | | | 15.5% | 17.6% | 40.8% | | 71.4% |
| 280+° C. | | | | 1.7% | 3.1% | 15.7% | | 27.5% |
| H2 | | | | | | | | 1.1% |
| H2O | 30% | 45.9% | | | | | | |
| Other | 1% | | | | | | | |
| Alcohols | | | | | | | | |
| Aldehydes | | 0.1% | | | | | | |

The entire disclosures of all applications, patents and publications, cited herein and of corresponding French Application No. 13/51886, filed Mar. 4, 2013 are incorporated by reference herein.

1. Process for the production of middle-distillate hydrocarbon-containing bases from a feedstock comprising butanol and pentanol, with said process comprising at least:
a) A stage for isomerizing dehydration of said feedstock in the presence of an amorphous or zeolitic acid catalyst in at least one reactor, operating at an absolute pressure at the reactor inlet of between 0.5 and 2 MPa and at a temperature at the reactor inlet of between 350 and 450° C. in such a way as to produce an effluent that is for the most part olefinic,
b) A stage for separation of the water that is present in said effluent that is for the most part olefinic operating at a pressure of between 0.5 and 1.2 MPa and at a temperature of between 35 and 60° C. in such a way as to obtain at least one organic liquid effluent,
c) A stage for purification of the organic liquid effluent coming from stage b) in such a way as to produce a purified organic effluent,
d) A first stage for selective oligomerization of isobutenes and isopentenes of a feedstock that comprises at least a portion of the purified organic effluent coming from stage c), in the presence of an amorphous catalyst in at least one reactor that operates at an absolute pressure of between 0.5 and 10 MPa, at a temperature at the reactor inlet of between 40 and 95° C., and at an hourly volumetric flow rate of between 0.1 and 10 h⁻¹, in such a way as to produce a first oligomerization effluent, comprising at least 20% by weight of olefins having a number of carbon atoms that is greater than or equal to 6, with the percentage by weight being expressed relative to the total mass of olefins contained in said effluent,
e) A second stage for oligomerization of said first oligomerization effluent, of the entire effluent coming from the oligomerization stage g), and at least a portion of the light product coming from the fractionation stage f), in the presence of an amorphous catalyst in at least one reactor operating at an absolute pressure of between 2 and 15 MPa, at a temperature at the reactor inlet of between 100 and 200° C., and at an hourly volumetric flow rate of between 0.1 and 10 h⁻¹, in such a way as to produce a second oligomerization effluent,
f) A stage for fractionation of said second oligomerization effluent into at least three products that correspond respectively to a light product that for the most part comprises the C₂ to C₅ compounds, an intermediate product that for the most part comprises the C₆ to C₉ compounds, and a middle distillate product that for the most part comprises the compounds that have at least 10 carbon atoms,
g) A third stage for oligomerization of at least a portion of said intermediate product in the presence of an amorphous catalyst in at least one reactor that operates at an absolute pressure of between 2 and 15 MPa, at a temperature of between 100 and 200° C., and at an hourly volumetric flow rate of between 0.1 and 5 h⁻¹,
h) A stage for hydrogenation of at least a portion of said middle distillate product in the presence of a catalyst that comprises at least one metal of group VIII in at least one reactor that operates at an absolute pressure of between 2 and 4 MPa, at a temperature of between 100 and 350° C., and at an hourly volumetric flow rate of between 1 and 5 h⁻¹ with a hydrogen to hydrocarbon H₂/HC molar ratio of between 10 and 450.

2. Process according to claim 1, in which a stage for purification of said feedstock comprising butanol and pentanol is carried out prior to stage a).

3. Process according to claim 1, in which the catalyst that is used in the dehydration stage a) is a zeolitic catalyst that comprises at least one zeolite that is selected from among the zeolites that have at least pore openings containing 10 or 12 oxygen atoms.

4. Process according to claim 1, in which the catalyst that is used in the dehydration stage a) is an amorphous acid catalyst that comprises at least one porous refractory oxide that is selected from among alumina, alumina activated by a deposition of mineral acid, and silica-alumina.

5. Process according to claim 1, in which said stage b) is a decanting stage in which an aqueous phase is separated from an organic phase.

6. Process according to claim 1, in which between 90 and 100% of the total mass flow rate of said intermediate product is treated in said oligomerization stage g).

7. Process according to claim 1, in which at least 90% of the total flow of the middle distillate product is treated in said hydrogenation stage h).

8. according to claim 1, in which said stage f) is carried out with two distillation columns operating in series.

9. Process according to claim 1, in which said stage f) is carried out with a column with an inside wall.

10. Process according to claim 1, in which a separation stage following the hydrogenation stage h) is carried out for making possible the fractionation into a kerosene fraction and/or a diesel fuel fraction and/or a fraction that has a boiling point of higher than 360° C. and/or light fractions.
